SEQUENCE LISTING (40 sequences)

SEQ ID NO: 1 - protein, 177 aa, synthetic construct
  REGION 1..177: note = amino acid sequence of human IL-7 (Accession number : P13232)
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 2 - protein, 154 aa, synthetic construct
  REGION 1..154: note = amino acid sequence of rat IL-7 (Accession number : P56478)
MFHVSFRYIFGIPPLILVLLPVTSSDCHIKDKDGKAFGSVLMISINQLDKMTGTDSDCPNNEPNFFKKHLCDDTKEAAFLNRAARKLRQFLKMNISEEFNDHLLRVSDGTQTLVNCTSKEEKTIKEQKKNDPCFLKRLLREIKTCWNKILKGSI

SEQ ID NO: 3 - protein, 154 aa, synthetic construct
  REGION 1..154: note = amino acid sequence of mouse IL-7 (Accession number : P10168)
MFHVSFRYIFGIPPLILVLLPVTSSECHIKDKEGKAYESVLMISIDELDKMTGTDSNCPNNEPNFFRKHVCDDTKEAAFLNRAARKLKQFLKMNISEEFNVHLLTVSQGTQTLVNCTSKEEKNVKEQKKNDACFLKRLLREIKTCWNKILKGSI

SEQ ID NO: 4 - protein, 177 aa, synthetic construct
  REGION 1..177: note = amino acid sequence of monkey IL-7 (Accession number : NP_001279008)
MFHVSFRYIFGLPPLILVLLPVASSDCDIEGKDGKQYESVLMVSIDQLLDSMKEIGSNCLNNEFNFFKRHLCDDNKEGMFLFRAARKLKQFLKMNSTGDFDLHLLKVSEGTTILLNCTGKVKGRKPAALGEPQPTKSLEENKSLKEQKKLNDSCFLKRLLQKIKTCWNKILMGTKEH

SEQ ID NO: 5 - protein, 176 aa, synthetic construct
  REGION 1..176: note = amino acid sequence of cow IL-7 (Accession number : P26895)
MFHVSFRYIFGIPPLILVLLPVASSDCDISGKDGGAYQNVLMVNIDDLDNMINFDSNCLNNEPNFFKKHSCDDNKEASFLNRASRKLRQFLKMNISDDFKLHLSTVSQGTLTLLNCTSKGKGRKPPSLSEAQPTKNLEENKSSKEQKKQNDLCFLKILLQKIKTCWNKILRGIKEH

SEQ ID NO: 6 - protein, 176 aa, synthetic construct
  REGION 1..176: note = amino acid sequence of sheep IL-7 (Accession number : Q28540)
MFHVSFRYIFGIPPLILVLLPVASSDCDFSGKDGGAYQNVLMVSIDDLDNMINFDSNCLNNEPNFFKKHSCDDNKEASFLNRAARKLKQFLKMNISDDFKLHLSTVSQGTLTLLNCTSKGKGRKPPSLGEAQPTKNLEENKSLKEQRKQNDLCFLKILLQKIKTCWNKILRGITEH

SEQ ID NO: 7 - protein, 384 aa, synthetic construct
  REGION 1..384: note = amino acid sequence of human IgD constant region (Genbank accession No. P01880)
APTKAPDVFPIISGCRHPKDNSPVVLACLITGYHPTSVTVTWYMGTQSQPQRTFPEIQRRDSYYMTSSQLSTPLQQWRQGEYKCVVQHTASKSKKEIFRWPESPKAQASSVPTAQPQAEGSLAKATTAPATTRNTGRGGEEKKKEKEKEEQEERETKTPECPSHTQPLGVYLLTPAVQDLWLRDKATFTCFVVGSDLKDAHLTWEVAGKVPTGGVEEGLLERHSNGSQSQHSRLTLPRSLWNAGTSVTCTLNHPSLPPQRLMALREPAAQAPVKLSLNLLASSDPPEAASWLLCEVSGFSPPNILLMWLEDQREVNTSGFAPARPPPQPGSTTFWAWSVLRVPAPPSPQPATYTCVVSHEDSRTLLNASRSLEVSYVTDHGPMK

SEQ ID NO: 8 - protein, 327 aa, synthetic construct
  REGION 1..327: note = amino acid sequence of Partial human IgG4 constant region (Genbank accession No. AAH25985)
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 9 - protein, 245 aa, synthetic construct
  REGION 1..245: note = amino acid sequence of hFc01
RNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 245 aa, synthetic construct
  REGION 1..245: note = amino acid sequence of hFc02
RNTGRGGEEKKGGKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 245 aa, synthetic construct
  REGION 1..245: note = amino acid sequence of hFc03
RNTGRGGEEKKGSKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - protein, 245 aa, synthetic construct
  REGION 1..245: note = amino acid sequence of hFc04
RNTGRGGEEKKSGKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 13 - protein, 245 aa, synthetic construct
  REGION 1..245: note = amino acid sequence of hFc05
RNTGRGGEEKKSSKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 243 aa, synthetic construct
  REGION 1..243: note = amino acid sequence of mouse IgG Fc variant
ASAEPRGPTIKPCPPCKCPAPNLEGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGNSGS

SEQ ID NO: 15 - protein, 153 aa, synthetic construct
  REGION 1..153: note = amino acid sequence of modified IL-7(M)
MDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 16 - protein, 154 aa, synthetic construct
  REGION 1..154: note = amino acid sequence of modified IL-7(MM)
MMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 17 - protein, 155 aa, synthetic construct
  REGION 1..155: note = amino acid sequence of modified IL-7(MMM)
MMMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 18 - protein, 155 aa, synthetic construct
  REGION 1..155: note = amino acid sequence of modified IL-7(MGM)
MGMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 19 - protein, 155 aa, synthetic construct
  REGION 1..155: note = amino acid sequence of modified IL-7(DDD)
DDDDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 20 - protein, 156 aa, synthetic construct
  REGION 1..156: note = amino acid sequence of modified IL-7(MMMM)
MMMMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 21 - protein, 398 aa, synthetic construct
  REGION 1..398: note = amino acid sequence of modified IL-7(M) fused hyFc
MDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 399 aa, synthetic construct
  REGION 1..399: note = amino acid sequence of modified IL-7(MM) fused hyFc
MMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 23 - protein, 400 aa, synthetic construct
  REGION 1..400: note = amino acid sequence of modified IL-7(MMM) fused hyFc
MMMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 400 aa, synthetic construct
  REGION 1..400: note = amino acid sequence of modified IL-7(MGM) fused hyFc
MGMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 25 - protein, 401 aa, synthetic construct
  REGION 1..401: note = amino acid sequence of modified IL-7(MMMM) fused hyFc
MMMMDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - protein, 397 aa, synthetic construct
  REGION 1..397: note = amino acid sequence of human IL-7 fused hyFc
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHRNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 395 aa, synthetic construct
  REGION 1..395: note = amino acid sequence of human IL-7 fused nonlytic mouse Fc
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHASAEPRGPTIKPCPPCKCPAPNLEGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGKGGGNSGS

SEQ ID NO: 28 - DNA, 531 bp, synthetic construct
  misc_feature 1..531: note = nucleotide sequence of human IL-7
  source 1..531: mol_type = other DNA
atgttccacgtgagcttcaggtacatcttcggcctgccacccctgatcctggtgctgctgcctgtggccagctccgactgcgacatcgagggaaaagacggcaagcagtacgaaagcgtgctgatggtgtccatcgaccagctgctggattctatgaaggagattgggagtaactgcctgaacaatgagttcaacttcttcaaacggcacatttgtgatgccaacaaggagggaatgttcctgtttcgggccgctagaaaactgaggcagttcctgaagatgaacagcaccggagactttgatctgcatctgctgaaagtgtctgagggcaccacaatcctgctgaactgcactgggcaggtgaaaggaaggaagcctgccgctctgggagaggctcagccaaccaagtcactggaggaaaacaaaagcctgaaggaacagaagaaactgaatgacctgtgctttctgaaacggctgctgcaggagatcaaaacatgttggaacaagattctgatgggcacaaaggaacac

SEQ ID NO: 29 - DNA, 534 bp, synthetic construct
  misc_feature 1..534: note = nucleotide sequence of modified IL-7(M)
  source 1..534: mol_type = other DNA
atgttccacgtgagcttcagatacatcttcggcctgccccccctgatcctggtgctgctgcccgtggccagcagcatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 30 - DNA, 537 bp, synthetic construct
  misc_feature 1..537: note = nucleotide sequence of modified IL-7(MM)
  source 1..537: mol_type = other DNA
atgttccacgtgagcttcagatacatcttcggcctgccccccctgatcctggtgctgctgcccgtggccagcagcatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 31 - DNA, 540 bp, synthetic construct
  misc_feature 1..540: note = nucleotide sequence of modified IL-7(MMM)
  source 1..540: mol_type = other DNA
atgttccacgtgagcttcagatacatcttcggcctgccccccctgatcctggtgctgctgcccgtggccagcagcatgatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 32 - DNA, 540 bp, synthetic construct
  misc_feature 1..540: note = nucleotide sequence of modified IL-7(MGM)
  source 1..540: mol_type = other DNA
atgttccacgtgagcttcaggtacatcttcggcctgccacccctgatcctggtgctgctgcctgtggccagctccatggggatggactgcgacatcgagggaaaagacggcaagcagtacgaaagcgtgctgatggtgtccatcgaccagctgctggattctatgaaggagattgggagtaactgcctgaacaatgagttcaacttcttcaaacggcacatttgtgatgccaacaaggagggaatgttcctgtttcgggccgctagaaaactgaggcagttcctgaagatgaacagcaccggagactttgatctgcatctgctgaaagtgtctgagggcaccacaatcctgctgaactgcactgggcaggtgaaaggaaggaagcctgccgctctgggagaggctcagccaaccaagtcactggaggaaaacaaaagcctgaaggaacagaagaaactgaatgacctgtgctttctgaaacggctgctgcaggagatcaaaacatgttggaacaagattctgatgggcaccaaggagcac

SEQ ID NO: 33 - DNA, 540 bp, synthetic construct
  misc_feature 1..540: note = nucleotide sequence of modified IL-7(DDD)
  source 1..540: mol_type = other DNA
atgttccacgtgagcttcagatacatcttcggcctgccccccctgatcctggtgctgctgcccgtggccagcagcgacgatgacgactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 34 - DNA, 543 bp, synthetic construct
  misc_feature 1..543: note = nucleotide sequence of modified IL-7(MMMM)
  source 1..543: mol_type = other DNA
atgttccacgtgagcttcagatacatcttcggcctgccccccctgatcctggtgctgctgcccgtggccagcagcatgatgatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcac

SEQ ID NO: 35 - DNA, 1284 bp, synthetic construct
  misc_feature 1..1284: note = nucleotide sequence of modified IL-7(M) fused hyFc
  source 1..1284: mol_type = other DNA
atgttccacgtgagcttcagatacatcttcggcctgccccccctgatcctggtgctgctgcccgtggccagcagcatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcacaggaacacaggcagaggcggcgaggagaagaagaaggagaaggagaaggaggagcaggaggaaagagagaccaagacccccgagtgccccagccacacccagcccctgggcgtgttcctgttccctcccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaagtgactcgagtctaga

SEQ ID NO: 36 - DNA, 1272 bp, synthetic construct
  misc_feature 1..1272: note = nucleotide sequence of modified IL-7(MM) fused hyFc
  source 1..1272: mol_type = other DNA
atgttccacgtgagcttcagatacatcttcggcctgccccccctgatcctggtgctgctgcccgtggccagcagcatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcacaggaacacaggcagaggcggcgaggagaagaagaaggagaaggagaaggaggagcaggaggaaagagagaccaagacccccgagtgccccagccacacccagcccctgggcgtgttcctgttccctcccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 37 - DNA, 1275 bp, synthetic construct
  misc_feature 1..1275: note = nucleotide sequence of modified IL-7(MMM) fused hyFc
  source 1..1275: mol_type = other DNA
atgttccacgtgagcttcagatacatcttcggcctgccccccctgatcctggtgctgctgcccgtggccagcagcatgatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcacaggaacacaggcagaggcggcgaggagaagaagaaggagaaggagaaggaggagcaggaggaaagagagaccaagacccccgagtgccccagccacacccagcccctgggcgtgttcctgttccctcccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 38 - DNA, 1275 bp, synthetic construct
  misc_feature 1..1275: note = nucleotide sequence of modified IL-7(MGM) fused hyFc
  source 1..1275: mol_type = other DNA
atgttccacgtgagcttcaggtacatcttcggcctgccacccctgatcctggtgctgctgcctgtggccagctccatggggatggactgcgacatcgagggaaaagacggcaagcagtacgaaagcgtgctgatggtgtccatcgaccagctgctggattctatgaaggagattgggagtaactgcctgaacaatgagttcaacttcttcaaacggcacatttgtgatgccaacaaggagggaatgttcctgtttcgggccgctagaaaactgaggcagttcctgaagatgaacagcaccggagactttgatctgcatctgctgaaagtgtctgagggcaccacaatcctgctgaactgcactgggcaggtgaaaggaaggaagcctgccgctctgggagaggctcagccaaccaagtcactggaggaaaacaaaagcctgaaggaacagaagaaactgaatgacctgtgctttctgaaacggctgctgcaggagatcaaaacatgttggaacaagattctgatgggcacaaaggaacaccgcaatactgggcggggcggggaggaaaagaaaaaggagaaggaaaaggaggaacaggaggaaagagagactaagaccccagaatgtcccagccatactcagcccctgggggtgttcctgtttccccctaaacctaaggataccctgatgatcagcaggacacccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 39 - DNA, 1278 bp, synthetic construct
  misc_feature 1..1278: note = nucleotide sequence of modified IL-7(MMMM) fused hyFc
  source 1..1278: mol_type = other DNA
atgttccacgtgagcttcagatacatcttcggcctgccccccctgatcctggtgctgctgcccgtggccagcagcatgatgatgatggactgcgacatcgagggcaaggacggcaagcagtacgagagcgtgctgatggtgagcatcgaccagctgctggacagcatgaaggagatcggcagcaactgcctgaacaacgagttcaacttcttcaagagacacatctgcgacgccaacaaggagggcatgttcctgttcagagccgccagaaagctgagacagttcctgaagatgaacagcaccggcgacttcgacctgcacctgctgaaggtgagcgagggcacaaccatcctgctgaactgcaccggccaggtgaagggcagaaagcccgccgccctgggcgaggcccagcccaccaagagcctggaggagaacaagagcctgaaggagcagaagaagctgaacgacctgtgcttcctgaagagactgctgcaggagatcaagacctgctggaacaagatcctgatgggcaccaaggagcacaggaacacaggcagaggcggcgaggagaagaagaaggagaaggagaaggaggagcaggaggaaagagagaccaagacccccgagtgccccagccacacccagcccctgggcgtgttcctgttccctcccaagcccaaggacaccctgatgatcagcagaacccccgaggtgacctgcgtggtcgtggatgtgagccaggaagatcccgaagtgcagttcaactggtacgtggatggcgtggaagtgcacaacgccaagaccaagcccagagaagagcagttcaactccacctacagagtggtgagcgtgctgaccgtgctgcaccaggactggctgaacggcaaggagtacaagtgcaaggtgtccaacaaaggcctgcccagctccatcgagaagaccatcagcaaagccaaaggccagcccagagaaccccaggtgtacaccctgcctcccagccaggaagagatgaccaagaaccaggtgtccctgacctgcctggtgaaaggcttctaccccagcgacatcgccgtggagtgggaaagcaacggccagcccgagaacaattacaagacaacccctcccgtgctggatagcgatggcagcttctttctgtacagcagactgaccgtggacaagagcagatggcaggaaggcaacgtgttcagctgcagcgtgatgcacgaagccctgcacaaccactacacccagaagagcctgtccctgagcctgggcaag

SEQ ID NO: 40 - protein, 4 aa, synthetic construct
  REGION 1..4: note = oligopeptides conjugated with IL-7
MMMM

(19) United States
(12) Patent Application Publication
(10) Pub. No.: US 2025/0257108 A1
(43) Pub. Date: Aug. 14, 2025

(54) PHARMACEUTICAL COMPOSITION COMPRISING IMMUNOGLOBULIN FC-FUSED INTERLEUKIN-7 FUSION PROTEIN FOR PREVENTING OR TREATING HUMAN PAPILLOMAVIRUS-CAUSED DISEASES

(71) Applicant: GENEXINE, INC., Seongnam-si (KR)

(72) Inventors: Moon Cheol KANG, Pohang-si (KR); Young Woo CHOI, Pohang-si (KR); Donghoon CHOI, Yongin-si (KR); Young Chul SUNG, Seoul (KR)

(73) Assignee: GENEXINE, INC., Seongnam-si (KR)

(21) Appl. No.: 19/028,190

(22) Filed: Jan. 17, 2025

Related U.S. Application Data
(63) Continuation of application No. 18/327,423, filed on Jun. 1, 2023.
Application No. 18/327,423 is a continuation of application No. 15/775,182, filed on May 10, 2018, now Pat. No. 11,708,399.
(60) Provisional application No. 62/361,170, filed on Jul. 12, 2016.
(60) Provisional application No. 62/360,696, filed on Jul. 11, 2016.
(60) Provisional application No. 62/263,262, filed on Dec. 4, 2015.

(51) Int. Cl. C07K 14/54; A61K 9/00; A61K 9/08; A61K 38/20; A61K 47/02; A61P 35/00

(52) U.S. Cl. CPC C07K 14/5418; A61K 9/0034; A61K 9/08; A61K 38/2046; A61K 47/02; A61P 35/00; C07K 2319/30

(57) ABSTRACT

The present invention relates to a pharmaceutical composition comprising an immunoglobulin Fc region and an IL-7 fusion protein. Specifically, when a fusion protein comprising the immunoglobulin Fc region and IL-7 is administered to an affected area, a strong immune response is induced in the body and thus allows human papillomavirus-caused diseases to be prevented or treated.

### TECHNICAL FIELD

The present invention relates to a composition of a fusion protein comprising interleukin-7 for preventing or treating a human papillomavirus-derived disease.

### BACKGROUND ART

Interleukin-7 (hereinafter ‘IL-7’) is an immune-stimulating cytokine that stimulates immune responses mediated by B cell and T cell, and plays an important role in the adaptive immune system. IL-7 is mainly secreted from stromal cells of bone marrow and thymus, but also produced in keratinocytes, dendritic cells, hepatocytes, nerve cells, and epithelial cells (Heufler C et al., 1993, J. Exp. Med. 178 (3)): 1109-14).

Specifically, interleukin-7 activates immune function through stimulation of the survival and differentiation of T cells and B cells, the survival of lymphoid cells, and the activation of NK (natural killer) cells, and is especially important for the development of T cells and B cells. It is bound with HGF (hepatocyte growth factor) and functions as pre-pro-B cell growth-stimulating factor or a cofactor for V (D) J rearrangement of T cell receptor beta (TCRβ) (Muegge K, 1993, Science 261 (5117): 93-5). In addition, interleukin-7 regulates lymph node development through lymphoid tissue inducer (LTi) cells and promotes the expansion and survival of naive T cells or memory T cells. It is also known that IL-7 stimulates the secretion of IL-2 and interferon-gamma (interferon-γ), thereby enhancing the human immune response.

Meanwhile, papillomavirus is a DNA-based virus with a diameter of 52 to 55 nm, which infects skin and subcutaneous tissue of humans and other animals. Human papillomavirus (HPV) is usually transmitted through skin keratinocytes or mucous membranes. More than 100 human papillomaviruses (HPV) have been found so far, most of which do not show any symptoms, but in some cases they can cause papillomas in humans. Some HPVs cause the development of warts, and some cause precancerous lesions. In particular, high-risk viruses such as human papilloma virus 16 (HPV 16) and human papilloma virus 18 (HPV 18) can cause cancer such as cervical cancer and testicular cancer.

Cervical cancer is one of the most common causes of cancer-related deaths in women worldwide. Almost all of the cases are caused by infection with human papillomavirus (HPV). Among them, HPV16 and HPV18 account for about 70-75% of cervical cancer patients. Continuous proliferation of infected cells leads to a pre-malignant cervical intraepithelial neoplasia (CIN), which then gradually transform into invasive cancer.

While the prophylactic HPV vaccines can efficiently prevent HPV infection, they do not have therapeutic effects against pre-existing infection and HPV-induced lesions. The most common treatment for CIN2 and CIN3 is surgical excision, which is associated with pregnancy-related complications and a 10% recurrence rate. More seriously, the mortality rate of cervical cancer after conventional treatment is more than 50%.

Meanwhile, recently, therapies to treat HPV infection have been developed by inducing immune enhancement. It has been reported that local administration of toll-like receptor (TLR) agent 7 and 9, imiquimod and CpG after administration of vaccine including HPV16 E7 antigen induced accumulation of E7-specific CD8 T cells in the genital tract and regression of genital tumors (Soong R-S et al., 2014, Clin. Cancer Res. 20:5456-67). However, in humans, imiquimod usage can induce side effects such as acute and severe local inflammation and ulceration, and administration of CpG requires repeated injections due to its short-lived efficacy. The ability of cytokines, such as IL-2 and IL-15, which function as vaccine adjuvants in animal models, were studied in order to enhance the therapeutic efficacy (Abraham E et al., 1992, J Immunol 149:3719-26). However, such cytokines also require repeated injections and may induce adverse effects, e.g., capillary leakage syndrome in case of IL-2.

Therefore, there still exists a need to develop effective and non-surgical therapy for the prevention and treatment of diseases caused by HPV infection.

### DISCLOSURE OF INVENTION

### Technical Problem

The object of the present invention is to provide a composition for preventing or treating a human papillomavirus-derived disease.

Another object of the present invention is to provide a method for preventing or treating a human papillomavirus-derived disease.

### Solution to Problem

In accordance with one aspect of the present invention, there is provided a pharmaceutical composition comprising a fusion protein of immunoglobulin Fc region and IL-7. Also, there is provided a method for preventing or treating a human papillomavirus-derived disease by mucosal administration of the pharmaceutical composition comprising the fusion protein.

### Advantageous Effects of Invention

In case where a fusion protein comprising immunoglobulin Fc region and IL-7 according to the present invention is administered via a mucosal route, the number of antigen-specific T cells is increased to prevent or treat a human papillomavirus-derived disease. Also, such administration is easy to conduct. Therefore, the fusion protein comprising immunoglobulin Fc region and IL-7 according to the present invention can be utilized as a new pharmaceutical composition which can replace the conventional HPV preventive vaccine.

### BRIEF DESCRIPTION OF DRAWINGS

FIG. 1 is a schematic illustration of the structure of IL-7 fused with Fc.

FIGS. 2a and 2b are bar graphs and fluorescence images, respectively, which show fluorescence intensities in various organs on days 1 and 7 after administration of Cy5.5 and IL-7-Fc-Cy5.5 to the mucous membrane, respectively (*, p<0.05).

FIG. 3 illustrates that IL-7-Fc is transported to serum through FcRn-mediated transcytosis after administration of PBS, rIL-7, and IL-7-Fc to the mice intravaginally (*, p<0.05 (rIL-7 vs IL-7-Fc)).

FIG. 4 shows the dot plot of the T cells, the number of CD4 and CD8 T cell counts, and the number of CD62^{low}CD44^{high} subsets in the CD4 and CD8 T cells (**, p<0.01), in cervical tissues.

FIG. 5 shows the results of T cell mobilization depending on IL-7-Fc administration route. At 7 days after vaginal administration, T cells in cervical (CV) tissues were analyzed by flow cytometry, and the numbers of CD4 T cells and CD8 T cells were counted (FIG. 5) (**, p<0.01).

FIG. 6 shows the results of observing the anticancer effect depending on the administration of IL-7-Fc.

### BEST MODE FOR CARRYING OUT THE INVENTION

Hereinafter, the present invention is explained in detail.

In one aspect for achieving the object, the present invention provides a pharmaceutical composition for preventing or treating a genital disease comprising an interleukin-7 (IL-7) fusion protein in which immunoglobulin Fc region is fused. The genital disease may be a human papillomavirus-derived disease.

As used herein, the term “human papillomavirus-derived disease” or “human papillomavirus infection disease” refers to a disease caused by human papilloma virus (HPV) infection. Human papilloma virus-derived diseases can be classified into CIN1, CIN2, CIN3, LSIL (low grade squamous intraepithelial lesion), HSIL (high grade squamous intraepithelial lesion) or cancer, etc., depending on the degree of infection or status of a lesion.

As used herein, the term “interleukin-7” may be a protein having the same amino acid sequence as interleukin-7 derived from an animal or a human. Further, the term “interleukin-7” may be a polypeptide or a protein having an activity similar to the interleukin-7 derived in vivo. Specifically, the IL-7 may be a protein comprising an IL-7 protein or a fragment thereof. Also, the IL-7 may be derived from a human, a rat, a mouse, a monkey, cattle or sheep.

The IL-7 comprises a polypeptide consisting of the amino acid sequences represented by SEQ ID NO: 1 to SEQ ID NO: 6. In addition, the IL-7 may have homology of about 70%, 75%, 80%, 85%, 90%, 91%, 92%, 93%, 94%, 95%, 96%, 97%, 98%, 99% or more to the sequences of SEQ ID NO: 1 to SEQ ID NO: 6.

Specifically, human IL-7 may have an amino acid sequence represented by SEQ ID NO: 1 (Genbank Accession No. P13232); rat IL-7 may have an amino acid sequence represented by SEQ ID NO: 2 (Genbank Accession No. P56478); mouse IL-7 may have an amino acid sequence represented by SEQ ID NO: 3 (Genbank Accession No. P10168); monkey IL-7 may have an amino acid sequence represented by SEQ ID NO: 4 (Genbank Accession No. NP_001279008); bovine IL-7 may have an amino acid sequence represented by SEQ ID NO: 5 (Genbank Accession No. P26895); and sheep IL-7 may have an amino acid sequence represented by SEQ ID NO: 6 (Genbank Accession No. Q28540).

In addition, the IL-7 protein or a fragment thereof may comprise a variety of modified proteins or peptides, i.e., variants. Such modification may be carried out by substitution, deletion or addition of one or more proteins of wild-type IL-7, which does not alter the function of IL-7. These various proteins or peptides may have homology of 70%, 75%, 80%, 85%, 90%, 91%, 92%, 93%, 94%, 95%, 96%, 97%, 98%, or 99% to a wild-type protein.

In general, substitution of a wild-type amino acid residue can be accomplished by substituting alanine or a conservative amino acid that does not affect the charge, polarity, or hydrophobicity of the entire protein.

The term “IL-7 protein” as used in the specification may be used as a concept including “IL-7 protein” and a fragment thereof. The terms “protein,” “polypeptide,” and “peptide” may be used interchangeably, unless otherwise specified.

In addition, the IL-7 may be a modified IL-7 having the following structure:
A-IL-7,
wherein said A is an oligopeptide consisting of 1 to 10 amino acid residues,
and the IL-7 is an interleukin-7 or a polypeptide having the activity similar to the interleukin-7.

Herein, said A may be directly linked to the N-terminus of the IL-7 or may be linked through a linker.

Said A may increase the productivity of IL-7 and may be prepared according to the method disclosed in Korean Patent Application No. 10-2016-0072769.

As used herein, said A may be linked to the N-terminus of IL-7. In the above formula, said A is characterized by containing 1 to 10 amino acids, which may be preferably selected from the group consisting of methionine, glycine, serine, and a combination thereof.

It is known that methionine and glycine do not induce an immune response in the human body. Although various protein therapeutic agents produced from E. coli necessarily contain methionine at the N-terminus thereof, no adverse immune effect has been reported. In the meantime, glycine is widely used in GS linker, and it is known that a commercial product such as Dulaglutide does not induce an immune response.

According to one embodiment, the IL-7 may be an oligopeptide comprising 1 to 10 amino acids selected from the group consisting of methionine (Met, M), glycine (Gly, G) and a combination thereof. Preferably, the IL-7 may be an oligopeptide consisting of 1 to 5 amino acids. For example, said A may be represented by the amino acid sequence selected from the group consisting of methionine, glycine, methionine-methionine, glycine-glycine, methionine-glycine, glycine-methionine, methionine-methionine-methionine, methionine-methionine-glycine, methionine-glycine-methionine, glycine-methionine-methionine, methionine-glycine-glycine, glycine-methionine-glycine, glycine-glycine-methionine, and glycine-glycine-glycine. Herein, the modified IL-7 may have any one of the amino acid sequences selected from SEQ ID NOS: 15 to 20.

Further, immunoglobulin Fc region may comprise an animal or human immunoglobulin Fc region, or a modified immunoglobulin Fc region thereof.

The IL-7 may be linked to the N-terminus or the C-terminus of the Fc region. It is known that even when IL-7 is fused to the C-terminus of the Fc region, IL-7 activity is maintained (U.S. Pat. No. 8,338,575 B2). Herein, the IL-7 may be linked to Fc region through a linker.

As used herein, the term “Fc region,” “Fc fragment” or “Fc” refers to a protein which comprises heavy chain constant region 2 (CH2) and heavy chain constant region 3 (CH3) of immunoglobulin but does not comprise variable regions of heavy or light chain and light chain constant region 1 (CL1). It may further comprise a hinge region of the heavy chain constant region. Hybrid Fc or a hybrid Fc fragment may herein also be referred to as “hFc” or “hyFc.” Also, as used herein, the term “a modified immunoglobulin Fc region” or “Fc region variant” refers to a Fc region in which one or more amino acids in the Fc region are substituted or a Fc region which is prepared by combining different Fc regions. Preferably, it refers to a Fc region whose binding force with a Fc receptor and/or a complement has been modified so as to exhibit weakened antibody-dependent cell-mediated cytotoxicity (ADCC) or complement dependent cytotoxicity (CDC) compared to the wild-type Fc region. The modified immunoglobulin Fc region may be a combination sequence of two or more of IgG1, IgG2, IgG3, IgD, and IgG4.

In particular, the modified immunoglobulin Fc region comprises CH2 domain and CH3 domain in the N-terminus to C-terminus direction, wherein the CH2 domain comprises a portion of an amino acid residue of CH2 domain of human IgD and human IgG4, and the CH3 domain comprises a portion of an amino acid residue of human IgG4 CH3 domain.

The Fc region variant can be modified so as to prevent the cleavage at the hinge region. Specifically, the 144^{th} amino acid and/or the 145^{th} amino acid of SEQ ID NO: 9 can be modified. Preferably, the variant may be a mutant in which K, the 144^{th} amino acid of SEQ ID NO: 9, is substituted by G or S, and E, the 145^{th} amino acid, is substituted by G or S.

In particular, the Fc region of the modified immunoglobulin comprises CH2 domain and CH3 domain in the N-terminus to C-terminus direction, wherein the CH2 domain comprises a portion of an amino acid residue of CH2 domain of human IgD and human IgG4, and the CH3 domain comprises a portion of an amino acid residue of human IgG4 CH3 domain.

As used herein, the term “Fc region”, “Fc fragment” or “Fc” refers to a protein which comprises heavy chain constant region 2 (CH2) and heavy chain constant region 3 (CH3) of immunoglobulin but does not comprise variable regions of heavy or light chain light chain and constant region 1 (CL1). It may further comprise a hinge region of the heavy chain constant region. Hybrid Fc or a hybrid Fc fragment may herein also be referred to as “hFc” or “hyFc”. Also, as used herein, the term “Fc region variant” refers to a Fc region in which one or more amino acids in the Fc region are substituted or which is produced by combining different Fc regions. The Fc region variant can be modified so as to prevent severing at the hinge region. Specifically, the 144^{th} amino acid and/or the 145^{th} amino acid of SEQ ID NO: 9 can be modified. Preferably, the variant may be a mutant in which K, the 144^{th} amino acid of SEQ ID NO: 9, is substituted by G or S, and E, the 145^{th} amino acid, is substituted by G or S.

In addition, the hFc can be represented by the following formula (I):

N′-(Z1)p-(Y)q-Z2-Z3-Z4-C′, [Formula (I)]
wherein,
N′ is the N-terminus of a polypeptide and C′ is the C-terminus of the polypeptide,
p or q is an integer of 0 or 1,
Z1 is an amino acid sequence having 5 to 9 consecutive amino acid residues in the N-terminus direction from the 98^{th} position in the amino acid residues at 90^{th} to 98^{th} positions of SEQ ID NO: 7,
Y is an amino acid sequence having 5 to 64 consecutive amino acid residues in the N-terminus direction from the 162^{th} position in the amino acid residues at 99^{th} to 162^{nd} positions of SEQ ID NO: 7,
Z2 is an amino acid sequence having 4 to 37 consecutive amino acid residues in the C-terminus direction from the 163^{rd} position in the amino acid residue at positions 163^{rd} to 199^{th} in SEQ ID NO: 7,
Z3 is an amino acid sequence having 70 to 106 consecutive amino acid residues in the N-terminus direction from the 220^{th} position in the amino acid residues at 115″ to 220^{th} positions of SEQ ID NO: 8, and
Z4 is an amino acid sequence having 80 to 107 consecutive amino acid residues in the C-terminus direction from the 221^{th} position in the amino acid residues at 221^{st} to 327^{th} positions of SEQ ID NO: 8.

In addition, the modified immunoglobulin Fc region or Fc region variant can be represented by the following formula (I):

N′-(Z1)p-Y-Z2-Z3-Z4-C′, [Formula (I)]
wherein,
N′ is the N-terminus of a polypeptide and C′ is the C-terminus of the polypeptide,
p is an integer of 0 or 1,
Z1 is an amino acid sequence having 5 to 9 consecutive amino acid residues in the N-terminus direction from the 988 position in the amino acid residues at 900 to 98^{th} positions of SEQ ID NO: 7,
Y is an amino acid sequence having 5 to 64 consecutive amino acid residues in the N-terminus direction from the 162^{th} position in the amino acid residues at 99^{th} to 162^{nd} positions of SEQ ID NO: 7,
Z2 is an amino acid sequence having 4 to 37 consecutive amino acid residues in the C-terminus direction from the 163^{rd} position in the amino acid residue at positions 163^{rd} to 199^{th} in SEQ ID NO: 7,
Z3 is an amino acid sequence having 70 to 106 consecutive amino acid residues in the N-terminus direction from the 220^{th} position in the amino acid residues at 115^{th} to 220^{th} positions of SEQ ID NO: 8, and
Z4 is an amino acid sequence having 80 to 107 consecutive amino acid residues in the C-terminus direction from the 221^{st} position in the amino acid residues at 221^{st} to 3270 positions of SEQ ID NO: 8.

In addition, Fc fragment of the present invention may be a wild type sugar chain, an increased sugar chain compared with the wild type, a reduced sugar chain compared with the wild type, or a form in which the sugar chain is removed. The increase, reduction or removal of immunoglobulin Fc sugar chain can be carried out by a conventional method known in the art such as chemical method, enzymatic method and genetic engineering method using microorganisms. The removal of the sugar chain from Fc fragment rapidly reduces the binding affinity of the primary complement component C1 to C1q and results in a decrease or loss of ADCC (antibody-dependent cell-mediated cytotoxicity) or CDC (complement-dependent cytotoxicity), thereby not inducing unnecessary immune responses in vivo. In this regard, immunoglobulin Fc fragment in a deglycosylated or aglycosylated form may be more suitable for the purpose of the present invention as a carrier of a drug. As used herein, the term “deglycosylation” refers to enzymatical elimination of sugar from Fc fragment, and the term “aglycosylation” refers to the production of Fc fragment in an unglycosylated form by a prokaryote, preferably E. coli.

The modified immunoglobulin Fc region may comprise amino acid sequences of SEQ ID NO: 9 (hFc01), SEQ ID NO: 10 (hFc02), SEQ ID NO: 11 (hFc03), SEQ ID NO: 12 (hFc04) or SEQ ID NO: 13 (hFc05). In addition, the modified immunoglobulin Fc region may comprise the non-lytic mouse Fc of SEQ ID NO: 14.

According to the present invention, the modified immunoglobulin Fc region may be one described in U.S. Pat. No. 7,867,491, and the production of the modified immunoglobulin Fc region may be carried out with reference to the disclosure of U.S. Pat. No. 7,867,491.

In addition, the interleukin-7 fusion protein in which immunoglobulin Fc region is fused may have the amino acid sequence of any one of SEQ ID NOS: 21 to 27.

Meanwhile, the interleukin-7 fusion protein in which immunoglobulin Fc region is fused according to the present invention may further comprise a pharmaceutically acceptable carrier. The pharmaceutically acceptable carrier may be any carrier that is suitable for being delivered to a patient and is non-toxic to the patient. Distilled water, alcohol, fats, waxes and inert solids may be included as carriers. Pharmacologically acceptable adjuvant (a buffer or a dispersant) may also be included in the pharmacological composition.

In another aspect of the present invention, there is provided a method for preventing or treating a genital disease comprising administering to an individual an interleukin-7 (IL-7) fusion protein in which immunoglobulin Fc region is fused and a pharmaceutically acceptable carrier.

The genital disease may be a human papillomavirus-derived disease, for example, cervical cancer.

Herein, the method of administration to an individual may be a local administration, preferably mucosal administration. In case of that the composition of the present invention is provided topically, such as intravaginal or aerosol administration, the composition preferably comprises a portion of an aqueous or physiologically compatible body fluid suspension or solution. Accordingly, the carrier or vehicle may be physiologically acceptable, and thus it can be added to the composition and delivered to the patient, which does not adversely affect the electrolyte and/or volume balance of the patient. Thus, a carrier for a formulation may generally include physiologic saline. Also, it may include a portion of viscous suspension or solution depending on the lesion or physiological condition.

The method for preventing or treating a disease using a fusion protein of the present invention or a composition comprising the same may comprise administering another drug or physiologically active substance having the effect of preventing or treating a disease in combination with the protein or the composition of the present invention, while the route, timing, and dosage of the administration may be determined depending on the type of a disease, the disease condition of a patient, the purpose of treatment or prevention, and other drugs or physiologically active substances co-administered.

The isolated nucleic acid molecule encoding the modified interleukin-7 or a fusion protein comprising the same may encode a polypeptide having an amino acid sequence selected from the group consisting of SEQ ID NOS: 15 to 25. The nucleic acid molecule may comprise a polynucleotide sequence selected from the group consisting of SEQ ID NOS: 29 to 39. The nucleic acid molecule may further comprise a signal sequence or a leader sequence.

### MODE FOR THE INVENTION

Hereinafter, the present invention is explained in detail. The following Examples are intended to further illustrate the present invention without limiting its scope.

### Preparation Example 1: Preparation of Experimental Animals

Female C57BL/6 mice, 8-10 weeks of age used in the following examples were purchased from The Jackson Laboratory (Bar Harbor, USA). All animals were raised under specific pathogen-free conditions in the animal care facility in POSTECH. The procedures of animal experiments were performed in accordance with the National Institutes of Health (NIH) guidelines for mouse experiments. The protocol was approved by the Institutional Animal Care and Use Committee (IACUC). Also, female Sprague-Dawley rats at 11 weeks of age were purchased from the Charles River Laboratories (Raleigh, USA). All animals were raised under specific pathogen-free conditions in the animal care facility of MPI research. The procedures of animal experiments were performed in accordance with the regulations outlined in the United States Department of Agriculture (USDA) animal welfare act (9 CFR, parts 1-3).

### Preparation Example 2: Preparation and Treatment of Fusion Protein of Fc and IL-7

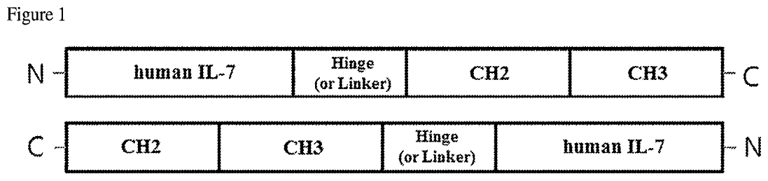

The codon-optimized human IL-7 and granulocyte colony-stimulating factor (G-CSF) genes were individually fused with a hybrid Fc-fragment. The schematic structure of Fc-fused IL-7 is shown in FIG. 1. Chinese hamster ovary (CHO) cells were stably transfected with a plasmid encoding IL-7-Fc and G-CSF-Fc. And then, IL-7-Fc and G-CSF-Fc were obtained from the cells. Purified recombinant human IL-7 (rIL-7), for a control group, was purchased from Biolegend (San Diego, USA).

3 mg of medroxyprogesterone acetate (Depo-Provera, Pfizer) was subcutaneously injected to mice in a diestrus state 4 days before treatment. The mice were anesthetized by intraperitoneal injection with 100 mg/kg ketamine (Yuhan) and 10 mg/kg xylazine hydrochloride (Bayer) in PBS. Then, 10 μg of rIL-7, IL-7-Fc or G-CSF-Fc were mixed with PBS and applied (administered) on the vaginal mucosal tissues using a micropipette.

### Preparation Example 3: Identification of Fluorescence-Conjugated IL-7-Fc in the Genital Tract

IL-7-Fc was coupled with Cy-5.5 mono-reactive NHS ester. Eluted proteins were desalted and concentrated by using centrifugal filter devices (Merck Millipore) and protein concentration of the dye-labeled IL-7-Fc was measured using an anti-human IL-7 ELISA set (Southern Biotech). Cy-5.5-conjugated IL-7-Fc (1 mg/kg) and Cy-5.5 in PBS were intravaginally administered to anesthetized mice with equivalent signal intensity. At days 1 and 7 after administration, mice were euthanized and their vaginas were washed, and each of the organs was obtained. The fluorescence signal intensity was then quantified using an IVIS spectral machine (Caliper Life Science). Signal intensity was measured quantitatively in the organ by measuring photons per second per centimeter squared per steradian (p/s/cm2/sr).

### Preparation Example 4: Quantification of Serum IL-7

Blood samples were collected before administration and up to 7 days after administration of IL-7-Fc, and serum IL-7 concentration was measured using a human IL-7 ELISA set (Southern biotech).

### Preparation Example 5: Toxicity Studies Depending on Repeated Administration

After topical administration of IL-7-Fc, for histopathological analysis using a microscope, 0.8, 3 and 8 mg/kg/dose of IL-7-Fc were intravaginally administered to rats once a week for 4 weeks (total dose of 5). The uterine cervix/vaginal tissues were excised and fixed with neutralizing formalin. The fixed tissues were placed in paraffin, cut with a thickness of 4-6 μm and stained with hematoxylin and eosin (H&E, Sigma-Aldrich). To determine the dose-dependence of vaginal inflammation, rats were observed individually at 4 hours and 24 hours after each dose administration and weekly. The following scoring scale was used: 0=no erythema, 1=very slight erythema (barely perceptible), 2=well-defined erythema, 3=moderate erythema, 4=severe erythema (redness) to eschar formation.

### Preparation Example 6: Splenocytes and Cervix/Vagina (CV) Cell Isolation

Spleen and CV tissues were surgically excised using sterile technique. The splenocytes were obtained by mechanically disrupting the tissues. For the preparation of CV cells, CV tissues were minced and treated with 1 mg/ml collagenase D (Roche) and 0.5 mg/ml DNase (Sigma-Aldrich). The cells were passed through a 40-μm strainer (BD), washed, and re-suspended with RPMI-1640 containing 10% FBS and antibiotics.

### Preparation Example 7: Flow Cytometry

To prevent non-specific binding of immunoglobulins to Fc receptor, the cells used in the following Examples were treated with CD16/32 (2.4G2) and stained with the following monoclonal antibodies: CD4 (RM4-5), CD8 (53-6.7), CD44 (IM7), CD62L (MEL-14), CD11b (M1/70), CD11c (N418), and MHCII (M5/114.15.2), from eBioscience; CD3e (145-2C1), and TCRY8 (GL3), from BD; CXCR3 (CXCR3-173), from Biolegend; and Live/Dead (Life technologies). All samples were analyzed using an LSR Fortessa (BD) and FlowJo software (Tree Star).

### Preparation Example 8: Statistical Analysis

A two-tailed paired Student's t-test was used to evaluate the statistical difference between the two experimental groups. For in vivo tumor experiments, differences in survival rates between the groups were determined by a log-rank test using the Prism 5.0 software (GraphPad).

### Example 1: Assessment of Administration Method of IL-7-Fc Fusion Protein

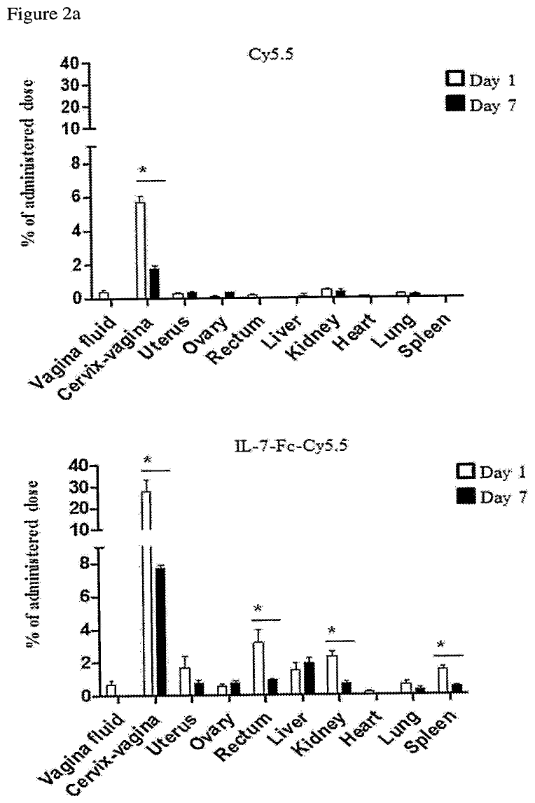

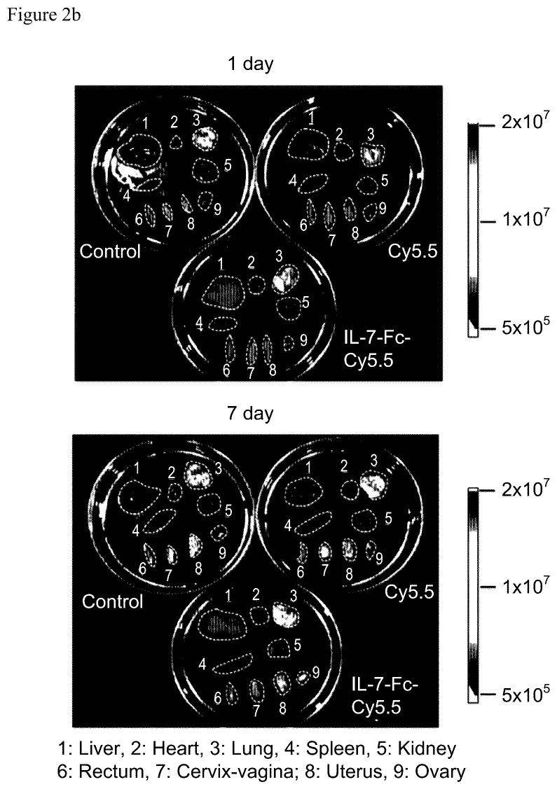

Cy-5.5 (Cy-5.5) and Cy-5.5-conjugated IL-7-Fc (Cy5.5-IL-7-Fc) were intravaginally administered to C57BL/6 wild-type mice (n=3/group). The results are shown in FIGS. 2a and 2b.

As shown in FIGS. 2a and 2b, the intensity of Cy-5.5-IL-7-Fc in the cervix/vagina (CV) tissues increased significantly at 1 day post-administration and observed for 7 days. In particular, signal intensities in CV tissues of Cy5.5-IL-7-Fc-treated mice were 6 and 4.5 times higher than the control (Cy5.5 treated mice) at days 1 and 7 after administration, respectively. Fluorescence signals were also detected at high intensities in various cervix/vagina adjacent tissues (cervix-vagina, uterus, ovary, and rectum) of Cy5.5-IL-7-Fc-treated mice. In particular, mice treated with Cy5.5-IL-7-Fc maintained high levels of fluorescence not only in the genital tract tissues but also in the liver, kidney and spleen even at day 7.

### Example 2: Confirmation of Systemic Circulation of Intravaginally Administered IL-7-Fc

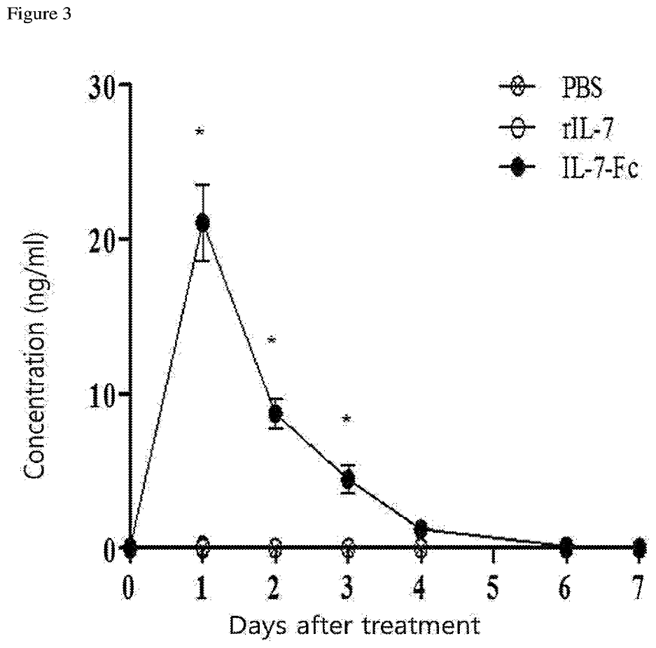

PBS, rIL-7 and IL-7-Fc were intravaginally administered to mice (n=7/group), and serum concentration of IL-7 was measured by human IL-7 ELISA. The results are shown in FIG. 3. As shown in FIG. 3, mice treated with IL-7-Fc, but not rIL-7, showed significantly increased levels of IL-7 as compared to PBS control.

These results reveal that the application of the Fc-fused protein on the mucosal epithelium enables genital-epithelial barrier transcytosis.

### Example 3: Analysis of Changes in Leukocyte Number in Cervical Tissues after Local Administration of IL-7-Fc

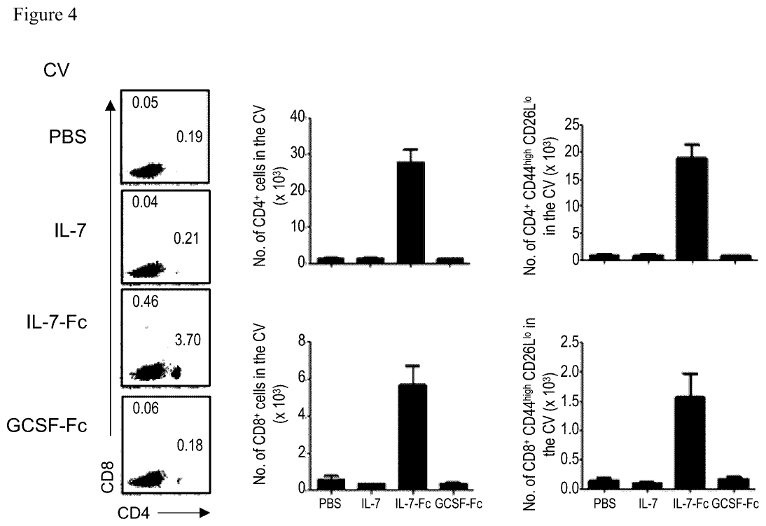

IL-7-Fc was intravaginally administered to mice (n=3/group) at 0, 3, 7, 14 and 21 days prior to sacrifice, and the number of leukocytes in cervical tissues was calculated using flow cytometry (Table 1). In addition, mice (n=6/group) were treated with PBS, IL-7, IL-7-Fc, IFN-α2a-Fc or G-CSF-Fc, and 7 days later, CD4 and CD8 T cells in CV tissues were analyzed by flow cytometry. The results are shown in Tables 1 and 2 and FIG. 4. The data in the table below are shown as means±SEMs (*, p<0.05).

TABLE 1

| | Absolute cell number after IL-7-Fc treatment | | | | |
|---|---|---|---|---|---|
| | Day 0 | Day 3 | Day 7 | Day 14 | Day 21 |
| Total CD4 T cells (×10³) | 2.86 ± 0.49 | 12.76 ± 0.53* | 51.51 ± 9.18* | 3.33 ± 0.77 | 2.57 ± 0.44 |
| CD62L^{lo} CD44^{high} CD4 T cells | 2.21 ± 40.31 | 10.26 ± 0.68* | 35.06 ± 7.03* | 2.51 ± 0.72 | 2.13 ± 0.41 |
| (×10³) | | | | | |
| Total CD8 T cells (×10³) | 0.49 ± 0.08 | 1.65 ± 0.18* | 6.21 ± 0.76* | 0.65 ± 0.17 | 0.84 ± 0.30 |
| CD62L^{lo} CD44^{high} CD8 T cells | 0.11 ± 0.01 | 0.64 ± 0.11* | 1.96 ± 0.29* | 0.23 ± 0.10 | 0.27 ± 0.14 |
| (×10³) | | | | | |
| γδ T cells (×10³) | 0.61 ± 0.14 | 2.40 ± 0.30* | 28.58 ± 3.88* | 2.05 ± 0.56* | 1.80 ± 0.07* |
| Conventional DC (×10³) | 0.33 ± 0.07 | 0.48 ± 0.09 | 2.15 ± 0.31* | 1.02 ± 0.12* | 0.56 ± 0.04 |
| Monocyte derived DC (×10³) | 4.78 ± 0.28 | 10.15 ± 0.83* | 38.89 ± 2.10* | 14.66 ± 2.16* | 5.64 ± 1.03 |

TABLE 2

| | 1 | 2 | 3 | 4 | 5 | Average ± STD |
|---|---|---|---|---|---|---|
| | % CD8 T cell in cervix/vagina | | | | | |
| PBS | 0.01 | 0.00 | 0.03 | 0.00 | 0.00 | 0.01 ± 0.01 |
| IL-7-Fc | 0.02 | 0.03 | 0.02 | 0.02 | 0.03 | 0.03 ± 0.01 |
| IFNα2a-Fc | 0.00 | 0.00 | 0.00 | 0.00 | 0.01 | 0.00 ± 0.00 |
| | % CD4 T cell in cervix/vagina | | | | | |
| PBS | 0.03 | 0.01 | 0.10 | 0.00 | 0.00 | 0.03 ± 0.04 |
| IL-7-Fc | 0.17 | 0.17 | 0.13 | 0.19 | 0.19 | 0.17 ± 0.03 |
| IFNα2a-Fc | 0.01 | 0.00 | 0.00 | 0.01 | 0.01 | 0.01 ± 0.00 |

As shown in Table 1 and FIG. 4, topical administration of IL-7-Fc increased the number of CD4 and CD8 T cells. This increase of genital tract T cells peaked at 7 days after IL-7-Fc administration and gradually decreased to the baseline levels at day 14. Moreover, the number of CD4 or CD8 T cells was significantly increased by about 20-fold and 10-fold, respectively, at 7 days after IL-7-Fc administration compared with the baseline levels. Particularly, the numbers of CD44^{high}CD62^{low} effector CD4 and CD8 T cells were significantly increased at day 7 and the number of total CD4 and CD8 T cells was decreased in a similar pattern over time.

As shown in Table 2 and FIG. 4, IFN-α2a-Fc, and G-CSF-Fc administration did not significantly change the number of CD4 and CD8 T cells compared to the baseline level or to the control group.

These results indicate that IL-7-Fc intravaginal administration induces local accumulation of immune cells such as T cells and DCs. Also, it was found that the effect of the IL-7-Fc intravaginal administration was superior to other immune inducers.

### Example 4: Evaluation of Toxicity of IL-7-Fc

IL-7-Fc was intravaginally administered to SD rats five times, i.e., at day 1, 8, 15, 22, and 29. Sections of the genital tract were microscopically examined at 33 days post-initial treatment (Table 3A). Vaginal inflammation scores were recorded prior to administration and at 4 and 24 hours after administration using the scoring scale (Table 3B). The results are shown in Tables 3A and 3B.

TABLE 3A

| | | | Dose (mg/kg) | | | |
|---|---|---|---|---|---|---|
| Tissue | Observation | Severity | 0 | 0.8 | 3 | 8 |
| Total | | | 10 | 10 | 10 | 10 |
| Ovaries | Mineralization^{a} | Minimal^{c} | 1 | 0 | 2 | 0 |
| | | Within normal limit^{e} | 9 | 10 | 8 | 10 |
| Uterus and | Infiltration^{b} | Minimal^{c} | 3 | 4 | 4 | 3 |
| Cervix | | Mild^{d} | 0 | 0 | 0 | 2 |
| | | Within normal limit^{e} | 7 | 6 | 6 | 5 |
| Vagina | Infiltration^{b} | Minimal^{c} | 4 | 3 | 3 | 6 |
| | | Mild^{d} | 0 | 0 | 0 | 1 |
| | | Within normal limit^{e} | 6 | 7 | 7 | 3 |

^{a}Mineralization: the formation or deposition of minerals in a tissue
^{b}Infiltration: the presence of mixed leukocyte (i.e. lymphocytes, dendritic cells, macrophage)
^{c}Minimal: the amount of change barely exceeds normal limits
^{d}Mild: easy identification of the lesion with limited severity and no functional impairment
^{e}Within normal limits: the condition to be considered normal

TABLE 3B

| Dose | | Study interval (Day) | | | | | | | | | | | | | | |
|---|---|---|---|---|---|---|---|---|---|---|---|---|---|---|---|---|
| (mg/kg) | Severity | 1^{a} | 1^{b} | 2^{c} | 8^{a} | 8^{b} | 9^{c} | 15^{a} | 15^{b} | 16^{c} | 22^{a} | 22^{b} | 23^{c} | 29^{a} | 29^{b} | 30^{c} |
| 0 | 0^{e} | 15 | 15 | 15 | 15 | 15 | 15 | 15 | 15 | 15 | 15 | 15 | 15 | 14 | 14 | 15 |
| (n^{d} = 15) | 1^{e} | 0 | 0 | 0 | 0 | 0 | 0 | 0 | 0 | 0 | 0 | 0 | 0 | 1 | 1 | 0 |
| | Total | 15 | 15 | 15 | 15 | 15 | 15 | 15 | 15 | 15 | 15 | 15 | 15 | 15 | 15 | 15 |
| 0.8 | 0^{e} | 10 | 10 | 10 | 10 | 10 | 10 | 10 | 10 | 10 | 10 | 10 | 10 | 10 | 10 | 10 |
| (n^{d} = 10) | Total | 10 | 10 | 10 | 10 | 10 | 10 | 10 | 10 | 10 | 10 | 10 | 10 | 10 | 10 | 10 |
| 3 | 0^{e} | 10 | 10 | 10 | 10 | 10 | 10 | 10 | 10 | 10 | 10 | 10 | 10 | 10 | 10 | 10 |
| (n^{d} = 10) | Total | 10 | 10 | 10 | 10 | 10 | 10 | 10 | 10 | 10 | 10 | 10 | 10 | 10 | 10 | 10 |
| 8 | 0^{e} | 15 | 15 | 15 | 15 | 15 | 15 | 15 | 15 | 14 | 15 | 15 | 15 | 15 | 15 | 15 |
| (n^{d} = 15) | 1^{e} | 0 | 0 | 0 | 0 | 0 | 0 | 0 | 0 | 1 | 0 | 0 | 0 | 0 | 0 | 0 |
| | Total | 15 | 15 | 15 | 15 | 15 | 15 | 15 | 15 | 15 | 15 | 15 | 15 | 15 | 15 | 15 |

^{a}predose
^{b}4 hour postdose
^{c}24 hour postdose
^{d}Number of mice
^{e}Vaginal imitation severity scoring scale: 0 = no erythema, 1 = very slight erythema (barely perceptible), 2 = well-defined erythema, 3 = moderate erythema, 4 = sever erythema (redness) to eschar formation

As shown in Tables 3A and 3B above, pathological evaluation of the degree of inflammation of cervical tissues (Table 3A) and vagina (Table 3B) showed that the local administration of IL-7-Fc was safe and did not induce serious inflammation within genital tract.

### Example 5: Confirmation of the Relationship Between the Administration Route of IL-7-Fc and the Induction of T Cells in the Cervix/Vaginal Tissues

IL-7-Fc was administered subcutaneously or intravaginally to mice (n=5/group) and the distribution of T cells in the cervix/vaginal tissues was observed by the method of Preparation Example 6.

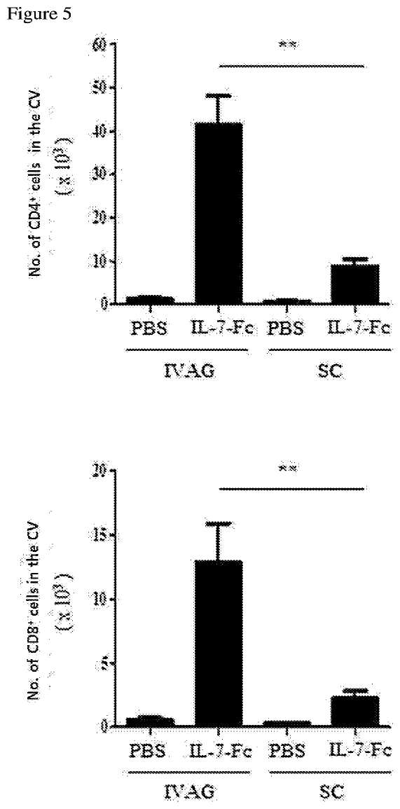

As a result, as shown in FIG. 5, the degree of accumulation of CD4 and CD8 T cells in the cervix/vaginal tissues was more increased by intravaginal administration than subcutaneous administration. Therefore, it was found that in order to induce CD4 and CD8 T cells specifically to the cervix/vaginal tissues, intravaginal administration which is directly related to the cervix/vaginal tissues is more effective than systemic administration such as subcutaneous administration.

### Example 6: Anticancer Efficacy by Local Administration of IL-7-Fc Using TC-1/Fluc Model

The therapeutic efficacy was confirmed using a TC-1 tumor cell line expressing HPV16 E6 and HPV E7 antigens. 1×10⁶ TC-1/fluc cell line (which was manipulated to express the luciferase gene in the TC-1 cell line expressing the HPV16 E6 and E7 gene) was administered intravaginally to the mice (n=7 or 8/group). Four (4) days before administration of the TC-1/fluc cell line, 3 mg of medroxyprogesterone acetate (Depo-Provera, Pfizer) was administered subcutaneously to the mice in the diestrus state. On the day of TC-1/fluc cell line administration, the mice were anesthetized and a mixture of 10 μl of 20% nonoxynol-9 (USP) and 40 μl of 3% carboxymethyl cellulose (CMC) (Sigma-Aldrich) was administered intravaginally to the mice, and 6 hours later, the mice were anesthetized again and their vaginas were washed with PBS and then TC-1/fluc cell line was administered to the mice.

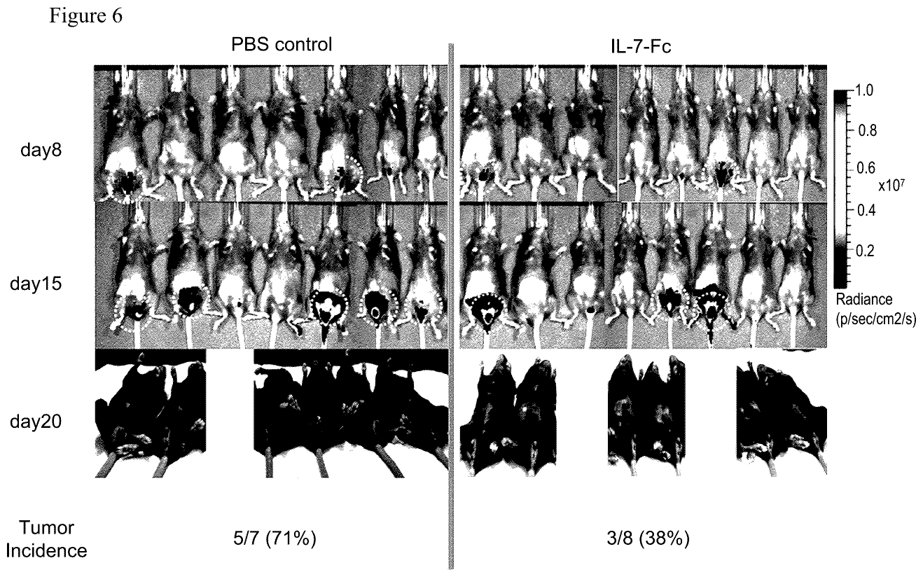

At 1, 8, and 15 days after TC-1/fluc cell line administration, 1 μg of IL-7-Fc was intravaginally administered to the mice, and the cancer progression was investigated by in vivo Bioluminescence imaging at days 8 and 15. At day 20, the anticancer effect was examined by observing the appearance (FIG. 6). As a result, it was confirmed that the incidence of cancer cells significantly decreased in the IL-7-Fc-treated group.

1. A pharmaceutical composition comprising an interleukin-7 (IL-7) fusion protein, wherein the IL-7 fusion protein comprises an IL-7 protein and an immunoglobulin Fc region.

2. The pharmaceutical composition of claim 1, wherein the IL-7 protein is conjugated to the N-terminus or C-terminus of the immunoglobulin Fc region.

3. The pharmaceutical composition of claim 1,
wherein the IL-7 fusion protein further comprises an oligopeptide.

4. The pharmaceutical composition of claim 1, wherein the IL-7 protein comprises an amino acid sequence which has at least about 70% sequence identity to the amino acid sequence set forth in any one of SEQ ID NOS: 1 to 6.

5. The pharmaceutical composition of claim 3, wherein the oligopeptide is selected from the group consisting of methionine, glycine, methionine-methionine, glycine-glycine, methionine-glycine, glycine-methionine, methionine-methionine-methionine, methionine-methionine-glycine, methionine-glycine-methionine, glycine-methionine-methionine, methionine-glycine-glycine, glycine-methionine-glycine, glycine-glycine-methionine, methionine-methionine-methionine, and glycine-glycine-glycine.

6-7. (canceled)

8. The pharmaceutical composition of claim 1, wherein the immunoglobulin Fc region comprises a CH2 domain and a CH3 domain in the N-terminus to C-terminus direction, wherein the CH2 domain comprises a portion of an amino acid residue of CH2 domain of human IgD and human IgG4, and the CH3 domain comprises a portion of an amino acid residue of human IgG4 CH3 domain.

9. The pharmaceutical composition of claim 1, wherein the immunoglobulin Fc region comprises the amino acid sequence set forth in any one of SEQ ID NOS: 9 to 14.

10. (canceled)

11. A method of preventing or treating a genital disease in a subject in need thereof comprising administering to the subject an interleukin-7 (IL-7) fusion protein, wherein the IL-7 fusion protein comprises an IL-7 protein and an immunoglobulin Fc region.

12-14. (canceled)

15. The pharmaceutical composition of claim 1, wherein the IL-7 protein does not comprise a signal peptide.

16. The pharmaceutical composition of claim 1, wherein the IL-7 protein comprises: (a) amino acid residues 26-177 of the amino acid sequence set forth in SEQ ID NO: 1, (b) amino acid residues 26-154 of the amino acid sequence set forth in SEQ ID NO: 2; (c) amino acid residues 26-154 of the amino acid sequence set forth in SEQ ID NO: 3; (d) amino acid residues 26-177 of the amino acid sequence set forth in SEQ ID NO: 4; (e) amino acid residues 26-176 of the amino acid sequence set forth in SEQ ID NO: 5; or (f) amino acid residues 26-176 of the amino acid sequence set forth in SEQ ID NO: 6.

17. The pharmaceutical composition of claim 1, wherein the IL-7 fusion protein comprises the amino acid sequence set forth in any one of SEQ ID NOs: 21-27.

18. A method of increasing an immune response within a mucosal tissue of a subject in need thereof, comprising administering to the subject the pharmaceutical composition of claim 1.

19. A modified interleukin-7 (IL-7) fusion protein comprising an IL-7 protein and an immunoglobulin Fc region.

20. The modified IL-7 fusion protein of claim 19, which further comprises an oligopeptide.

21. The modified IL-7 fusion protein of claim 19, wherein the IL-7 protein does not comprise a signal peptide.

22. The modified IL-7 fusion protein of claim 19, wherein the IL-7 protein comprises: (a) amino acid residues 27-177 of the amino acid sequence set forth in SEQ ID NO: 1, (b) amino acid residues 26-154 of the amino acid sequence set forth in SEQ ID NO: 2; (c) amino acid residues 26-154 of the amino acid sequence set forth in SEQ ID NO: 3; (d) amino acid residues 26-177 of the amino acid sequence set forth in SEQ ID NO: 4; (e) amino acid residues 26-176 of the amino acid sequence set forth in SEQ ID NO: 5; or (f) amino acid residues 26-176 of the amino acid sequence set forth in SEQ ID NO: 6.

23. The modified IL-7 fusion protein of claim 19, which comprises the amino acid sequence set forth in any one of SEQ ID NOs: 21-27.

24. The modified IL-7 fusion protein of claim 20, wherein the oligopeptide is selected from the group consisting of methionine, glycine, methionine-methionine, glycine-glycine, methionine-glycine, glycine-methionine, methionine-methionine-methionine, methionine-methionine-glycine, methionine-glycine-methionine, glycine-methionine-methionine, methionine-glycine-glycine, glycine-methionine-glycine, glycine-glycine-methionine, methionine-methionine-methionine, and glycine-glycine-glycine.

25. A method of producing the modified IL-7 fusion protein of claim 19, comprising transforming a cell with a polynucleotide comprising a first nucleic acid sequence encoding the IL-7 protein and a second nucleic acid sequence encoding the immunoglobulin Fc region, and culturing the cell after the transforming such that the IL-7 fusion protein is produced.

26. The method of claim 25, wherein the first nucleic acid sequence is codon-optimized.
